(19) United States
(12) Patent Application Publication
(10) Pub. No.: US 2009/0004066 A1
(43) Pub. Date: Jan. 1, 2009

(54) Air purifier with a sensor and/or timer switching function.

(76) Inventor: Yu-Lin Cheng, (US)

Correspondence Address: Yu-Lin Cheng, 9F, No. 20, Lane 50, Tian-Mu West Road, Taipei, TW

(21) Appl. No.: 11/772,121

(22) Filed: Jun. 30, 2007

(51) Int. Cl. A61L 9/20; A61L 9/00

(52) U.S. Cl. 422/121; 422/120

(57) ABSTRACT

An air purifier with a sensor and/or timer switching function is proposed, which comprises: a photo sensor or motion detecting sensor and /or timer switching circuit to automatically activate or turn off or reduce the lighting intensity of lamp in the apparatus when ambient lighting or motion detecting signal is changed, or by programmed time period to reduce the possibility of harmful lighting like UV to the human directly.

### FIELD OF THE INVENTION

The present invention relates to an air purifier for sanitizing by photocatalyst such as Titanium dioxide (TiO2) and UV lamp or visible light lamp with a photo sensor or motion detecting sensor and/or timer switching function to automatically activate or turn off or adjust the lighting intensity of lamp when ambient lighting or motion detecting signal is changed, or by programmed time period to reduce the possibility of harmful lighting like UV to the human directly.

### BACKGROUND OF THE INVENTION

FIG. 1 is a diagram of the mechanism of photocatalyst reaction. When photocatalyst such as Titanium dioxide (TiO2) activated by ultraviolet (UV) radiation (wavelength <380 nm) from sunlight or illuminated light source (fluorescent lamps) or visible light (wavelength >380 nm), it will produce pairs of electrons and holes. The electron of the valence band of photocatalyst becomes excited when illuminated by light. The excess energy of this excited electron promoted the electron to the conduction band of photocatalyst therefore creating the negative-electron (e−) and positive-hole (h+) pair. This stage is referred as the semiconductor's ‘photo-excitation’ state. The energy difference between the valence band and the conduction band is known as the ‘Band Gap’.

The positive-hole of photocatalyst breaks apart the water molecule to form hydrogen gas and hydroxyl radical. The negative-electron reacts with oxygen molecule to form super oxide anion. This strong oxidizing effect cycle continues when light is available.

Photocatalyst does not only kill bacteria cells, but also decompose the cell itself. The titanium dioxide photocatalyst has been found to be more effective than any other antibacterial agent, because the photocatalytic reaction works even when there are cells covering the surface and while the bacteria are actively propagating. The end toxin produced at the dead cell is decomposed by photocatalytic action. Titanium dioxide does not deteriorate and it shows a long-term anti-bacterial effect. Generally speaking, disinfections effect of titanium oxide is three times stronger than chlorine, and 1.5 times stronger than ozone.

On the deodorizing application, the hydroxyl radicals accelerate the breakdown of any Volatile Organic Compounds or VOCs by destroying the molecular bonds. This will help combine the organic gases to form a single molecule that is not harmful to humans thus enhance the air cleaning efficiency. Some of the examples of odor molecules are: Tobacco odor, formaldehyde, nitrogen dioxide, urine and fecal odor, gasoline, and many other hydrocarbon molecules in the atmosphere. Air purifier with TiO2 can prevent smoke and soil, pollen, bacteria, virus and harmful gas as well as seize the free bacteria in the air by filtering percentage of 99.9% with the help of the highly oxidizing effect of photocatalyst (TiO2).

Once the air purifier's power is turn on, the photo sensor or motion sensor switching circuit will react by the ambient light or motion signal changed to turn on or turn off or adjust the lighting intensity of lamp to activate or to disable or to limit the photocatalyst effect.

To put the air purifier into a closed space such as the light off toilet which is desired for disinfections, the lamp will be turn on by the dark atmosphere through the photo sensor circuit, or by the still atmosphere through the motion detecting sensor to activate the photocatalyst effect, and the lamp will be turn off or adjust the lighting intensity immediately or within a programmed time period by the bright atmosphere through the photo sensor circuit or by the motion signal changed through the motion detecting sensor when people switch on the light and enter the toilet to prevent the possibility of harmful lighting like UV to the human directly.

In addition, to apply the air purifier into a closed space such as car cabinet which is desired for disinfections, the lamp will be turn on and turn off by timer circuit to activate or to disable the photocatalyst effect by programmed time period.

### SUMMARY OF THE INVENTION

The air purifier of the present invention is provided a photo sensor or motion detecting sensor and/or timer switching function to limit or to disable the possibility of harmful lighting like UV to the human directly.

### BRIEF DESCRIPTION OF THE DRAWINGS

FIG. 1 is a diagram of the mechanism of photocatalyst reaction.

FIG. 2 is a front perspective view of external appearance of the apparatus of the first preferred embodiment of the present invention.

FIG. 3 is a right side plan view of the external appearance of the second preferred embodiment of the present invention.

FIG. 4 is a top view of the external appearance of the second preferred embodiment of the present invention.

FIG. 5 is a front view of external appearance of the apparatus of the third preferred embodiment of the present invention.

FIG. 6 is a car adapter for car application.

FIG. 7 is a front perspective view of external appearance of the apparatus of the fourth preferred embodiment of the present invention.

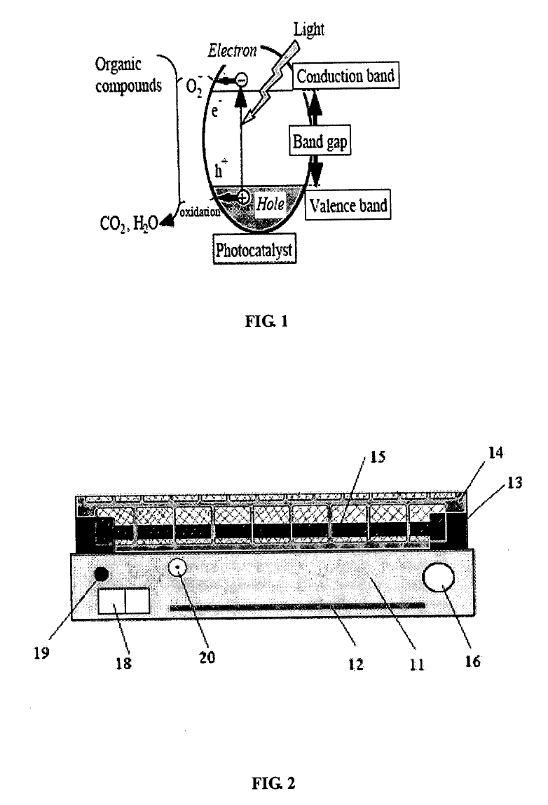

### DETAILED DESCRIPTION OF THE INVENTION

FIG. 2 is a front perspective view of external appearance of the air purifier of the first preferred embodiment of the present invention. A housing 11 for installing one inverter 12 to ignite the lamp 15 with photo sensor 16 switching circuit, the housing attaching one lamp 15 fixed by lamp holder 13 and a net 14 covered on the top of the lamp 15 which be sprayed with photocatalyst.

When AC to DC adapter (not shown) connects to plug receiver 20, switch on through power switch 18 and to light the LED 19 on. The lamp will be turn on by dark atmosphere through the photo sensor circuit to activate the photocatalyst effect, and turn off or adjust the lighting intensity by bright atmosphere to prevent the possibility of harmful lighting like UV to the human directly.

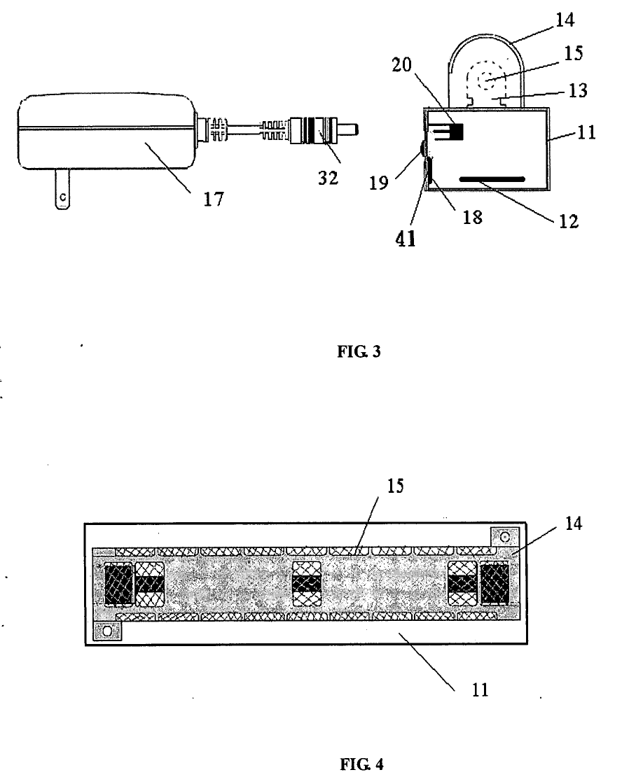

FIG. 3 is a right side perspective view of external appearance of the air purifier of the second preferred embodiment of the present invention. A housing 11 for installing one inverter 12 to ignite the lamp 15 with motion detecting sensor 41 switching circuit, the housing attaching one lamp 15 fixed by lamp holder 13 and a net 14 covered on the top of the lamp 15 which be sprayed with photocatalyst.

When AC to DC adapter 17 with plug 32 connects to plug receiver 20, switch on through power switch 18 and to light the LED 19 on. The lamp will be turn on by still atmosphere through the motion detecting sensor 41 switching circuit to activate the photocatalyst effect, and turn off or adjust the lighting intensity immediately or within a programmed time period by motion signal changed to prevent the possibility of harmful lighting like UV to the human directly.

FIG. 4 is a top view of external appearance of the air purifier of the second preferred embodiment of the present invention. A housing 11 for installing one inverter 12 to ignite the lamp 15 with motion detecting sensor 41 switching circuit, the housing attaching one lamp 15 fixed by lamp holder 13 and a net 14 covered on the top of the lamp 15 which be sprayed with photocatalyst.

FIG. 5 is a front view of external appearance of the air purifier of the third preferred embodiment of the present invention. A housing 11 for installing one AC to DC adapter 17 and one inverter 12 to ignite the lamp 15 with photo sensor 16 switching circuit, the housing attaching one lamp 15 fixed by lamp holder 13 and a net 14 covered on the top of the lamp 15 which be sprayed with photocatalyst.

When plug AC to DC adapter 17 into the city electricity and switch on the air purifier through power switch 18 to light the LED 19 on. The lamp will be turn on by dark atmosphere through the photo sensor circuit to activate the photocatalyst effect, and turn off or adjust the lighting intensity by bright atmosphere to prevent the possibility of harmful lighting like UV to the human directly.

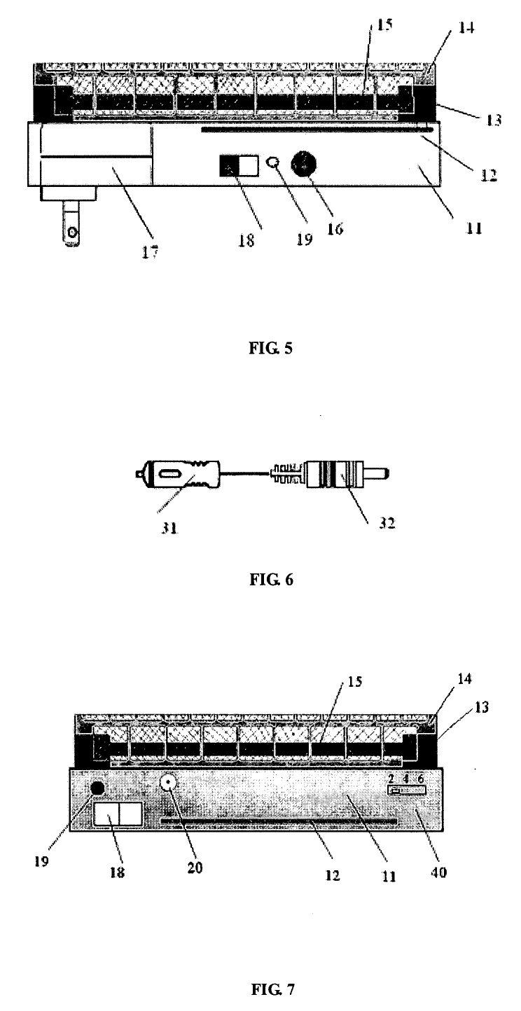

FIG. 6 is a view of external appearance of the car adapter 31 and plug 32 for automobile application.

FIG. 7 is a front perspective view of external appearance of the apparatus of the fourth preferred embodiment of the present invention. A housing 11 for installing one inverter 12 to ignite the lamp 15 with timer circuit 40 switching circuit, the housing attaching one lamp 15 fixed by lamp holder 13 and a net 14 covered on the top of the lamp 15 which be sprayed with photocatalyst.

When AC to DC adapter (not shown) connects to plug receiver 20, switch on through power switch 18 to light the LED 19 on and the lamp 15 will be turn on by timer circuit to activate the photocatalyst effect, and turn off by time setting.

While the preferred embodiment and various alternative embodiments of the invention have been disclosed and described in detail herein, it may be apparent to those skilled in the art that various changes in form and detail may be made therein without departing from the spirit and scope thereof.

### 11. Housing

### 12. Inverter

13. Lamp holder

### 14. Net

### 15. Lamp

16. Photo sensor
17. AC to DC adapter
18. power switch

### 19. LED

20. Plug receiver
31. Car adapter

### 32. Plug

40. Timer circuit
41. Motion detecting sensor

I claim:

1. An air purifier, which comprises: a housing for installing at least one power circuit to ignite the lamp with a sensor and/or timer switching circuit, the housing attaching at least one lamp; and a net covered on the top of the lamp which be coated or sprayed with photocatalyst.

2. The air purifier of claim 1, wherein the sensor is a photo sensor or motion detecting sensor.

3. The air purifier of claim 1, wherein the power circuit to ignite the lamp is a DC to AC inverter with adapter or an AC to AC inverter or a ballast.

4. The air purifier of claim 1, wherein the lamp is an Ultraviolet (UV) or visible light fluorescent lamp that is coated or sprayed with or without photocatalyst.

5. The air purifier of claim 3, wherein the adapter is a car adapter for automobile battery system or an AC to DC power adapter.

6. An air purifier, which comprises: a housing for installing at least one power circuit to ignite the lamp with a sensor and/or timer switching circuit, the housing attaching at least one lamp; and a net covered on the top of the lamp.

7. The air purifier of claim 6, wherein the lamp is an Ultraviolet (UV) or visible light fluorescent lamp that is coated or sprayed with photocatalyst outside.
